(19) United States
(12) Patent Application Publication
(10) Pub. No.: US 2025/0108065 A1
(43) Pub. Date: Apr. 3, 2025

(54) METHODS OF TREATING PREDIABETES OR REDUCING THE RISK OF DEVELOPING TYPE 2 DIABETES WITH DAPAGLIFLOZIN

(71) Applicant: AstraZeneca AB, Södertälje (SE)

(72) Inventors: Richard Skelly, Gaithersburg, MD (US); Howard Hutchinson, Gaithersburg, MD (US); Anna Maria Langkilde, Södertälje (SE)

(21) Appl. No.: 18/833,007

(22) PCT Filed: Jan. 25, 2023
(86) PCT No.: PCT/IB2023/050633

§ 371 Date: Jul. 25, 2024

Related U.S. Application Data
(60) Provisional application No. 63/267,179, filed on Jan. 26, 2022.

(51) Int. Cl. A61K 31/70; A61K 45/06; A61P 3/10; A61P 9/12

(52) U.S. Cl. CPC A61K 31/70; A61K 45/06; A61P 3/10; A61P 9/12

(57) ABSTRACT

The present disclosure is directed to methods of reducing the risk of developing Type 2 diabetes and/or treating prediabetes in a patient in need thereof, the method comprising administering to the patient, an effective amount of a sodium-glucose co-transporter 2 (SGLT2) inhibitor, wherein the patient has an HbA1c between 5.7% to 6.4% and/or a fasting glucose between 100 to 125 mg/dl.

Prediabetes is a serious health condition that increases the risk of Type 2 diabetes mellitus (T2D), micro- and macrovascular complications including CV, and kidney complications. In 2021, 10.6% of the global adult population (541 million people) were estimated to have prediabetes; the prevalence was projected to increase to 11.4% of the global population (730 million people) by 2045 (International Diabetes Federation. IDF Diabetes Atlas 2021). In the US, there are more people living with prediabetes than diabetes (88 million adults vs. 34 million adults) (Centers for Disease Control and Prevention. Diabetes and Prediabetes 2020).

Despite the seriousness of the condition, the vast majority (90%) of affected persons are unaware that they have prediabetes. As the person progresses to T2D, the risk for complications substantially increases. In 2021, an estimated 6.7 million deaths occurred worldwide in adults aged 20 to 79 years as a result of diabetes and its complications (International Diabetes Federation. IDF Diabetes Atlas 2021).

In the US, no drug products are approved for the treatment of prediabetes. Lifestyle changes, including adjustment to diet and exercise, are recommended for individuals with prediabetes. (American Diabetes Association. 3. Prevention or delay of type 2 diabetes. Diabetes Care. 2021; 44 (Suppl 1): S34-9.) The National Diabetes Prevention Program was created to address the increasing burden of prediabetes and T2D in the US. (Centers for Disease Control and Prevention. Diabetes and Prediabetes 2020.) A key message of the National Diabetes Prevention Program is that prediabetes can be reversed by taking an active role and engaging in long-term lifestyle change programs including a healthy diet and physical activity. However, lifestyle changes are difficult to maintain long term. (Diabetes Prevention Program Research Group 2009.)

Taken together, the ADA recognizes the difficulties in maintaining lifestyle changes and recommends pharmacologic therapy (e.g., metformin) for the prevention of T2D. (American Diabetes Association. 3. Prevention or delay of type 2 diabetes. Diabetes Care. 2021; 44 (Suppl 1): S34-9.) However, it is also recognized that long-term side effects and safety are important considerations (e.g., gastrointestinal intolerance and vitamin B12 deficiency from extended metformin use) necessitating other options. (Id.; Bonnet F and Scheen A. Understanding and overcoming metformin gastrointestinal intolerance. Diabetes Obes Metab. 2017; 19 (4): 473-81.)

Hence, there is an unmet need in the US from an individual and a societal perspective for pharmacological options to reduce the risk of progression to T2D and to reduce the risk of micro- and macrovascular complications in people who have prediabetes. Dapagliflozin is a potent, highly selective and orally active inhibitor of human renal SGLT2. The chemical structure of dapagliflozin is:

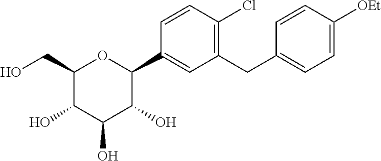 [SMILES: CCOc1ccc(Cc2cc([C@@H]3O[C@H](CO)[C@@H](O)[C@H](O)[C@H]3O)ccc2Cl)cc1]

The present disclosure is directed to methods of reducing the risk of developing Type 2 diabetes and methods of treating prediabetes with an SGLT2 inhibitor, e.g., dapagliflozin.

### SUMMARY OF THE DISCLOSURE

The present disclosure relates to methods of reducing the risk of developing Type 2 diabetes in a patient in need thereof. In some embodiments, the method comprises administering to the patient an effective amount of a sodium-glucose co-transporter 2 (SGLT2) inhibitor, wherein the patient has an HbA1c between 5.7% to 6.4% and/or a fasting glucose between 100 to 125 mg/dl.

Also disclosed are methods of treating prediabetes in a patient in need thereof. In some embodiments, the method comprises administering to the patient an effective amount of a SGLT2 inhibitor, wherein the patient has an HbA1c between 5.7% to 6.4% and/or a fasting glucose between 100 to 125 mg/dl.

In some embodiments of the disclosed methods herein, the patient has Type 1 diabetes (T1D). In some embodiments, the patient has Type 2 diabetes (T2D). In some embodiments, the patient does not have T1D or T2D. In some embodiments, the patient was not previously administered a prescription medicine for diabetes. In some embodiments, the patient does not have chronic kidney disease (CKD) and/or heart failure (HF).

In any of the embodiments disclosed herein, the SGLT2 inhibitor is dapagliflozin, canagliflozin, empagliflozin, sotagliflozin, ipragliflozin, or ertugliflozin, or a pharmaceutically acceptable salt, solvate, mixed solvate, complex, or prodrug thereof. In at least one embodiment, the SGLT2 inhibitor is dapagliflozin, or a pharmaceutically acceptable salt, solvate, mixed solvate, complex, or prodrug thereof. In at least one embodiment, dapagliflozin is in the form of a non-crystalline solid. In at least one embodiment, dapagliflozin is in the form of a crystalline solid. In at least one embodiment, dapagliflozin is in the form of a(S)-propylene glycol ((S)-PG) solvate, which has the structure

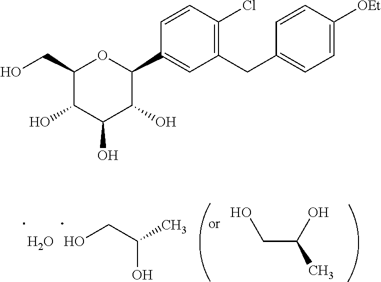

Further disclosed herein are methods comprising administering to a patient in need thereof an effective amount of a SGLT2 inhibitor alone or in combination with at least one other therapeutic agent. In some embodiments, the other therapeutic agent is administered with the SGLT2 inhibitor in the same or in different pharmaceutical compositions. In embodiments in which the other therapeutic agent is administered with the SGLT2 inhibitor in different pharmaceutical compositions, the SGLT2 inhibitor and the other therapeutic agent are administered at the same or at a different time. In some embodiments, the other therapeutic agent is an antidiabetic agent, anti-obesity agent, anti-hyperlipidemic agent, anti-atherosclerotic agent, anti-hypertensive agent, anti-platelet agent, antithrombotic agent, or anticoagulant agent. In some embodiments, the other therapeutic agent is an angiotensin-converting enzyme inhibitor (ACE-I). In some embodiments, the other therapeutic agent is an angiotensin receptor blocker (ARB). In some embodiments, the ACE-I is chosen from captopril, enalapril, and lisinopril. In some embodiments, the ARB is chosen from valsartan, losartan, and irbesartan.

In some embodiments, the methods disclosed herein comprise orally administering to the patient an SGLT2 inhibitor, such as dapagliflozin or a pharmaceutically acceptable salt, solvate, mixed solvate, complex, or prodrug thereof, at a dose of 2.5 mg, 5.0 mg, or 10 mg, once a day. In at least one embodiment, the dose is 2.5 mg. In at least one embodiment, the dose is 5 mg. In some embodiments, the SGLT2 inhibitor, such as dapagliflozin is in the form of a tablet.

In some embodiments of the methods disclosed herein, the patient had an eGFR of ≥39 and ≤67 mL/min/1.73 m² prior to the administration. For example, in some embodiments, the patient had an eGFR of ≥60 mL/min/1.73 m² prior to the administration.

In some embodiments, the methods disclosed herein result in a relative risk reduction of 25% for developing T2D. In some embodiments, the methods disclosed herein result in a relative risk reduction of 30% for developing T2D. In some embodiments, the methods disclosed herein results in an absolute risk reduction of 3% or more for developing T2D during a period of 1.75 years. At least in some embodiments, the method results in a hazard ratio from 0.65 to 0.8. For example, in some embodiments, the methods disclosed herein result in a hazard ratio of 0.75, 0.72 or 0.69.

In some embodiments of the methods disclosed herein, the patient satisfies at least one of the following conditions:
(a) the patient has a body mass index (BMI) of ≥30 kg/m²;
(b) the patient has a first degree relative with T2D;
(c) the patient has a medical history of hypertension;
(d) the patient has a medical history of dyslipidemia;
(e) the patient has prior gestational diabetes; and/or
(f) the patient has polycystic ovary syndrome.

In some embodiments of the disclosed methods herein, the patient has a BMI of ≥25 kg/m². For example, in some embodiments, the patient has a BMI of ≥30 kg/m².

In some embodiments of the disclosed methods herein, the patient is ≥45 years old and has a body mass index of ≥30 kg/m² for non-Asians or >27 kg/m² for Asians. In some embodiments of the methods disclosed herein, the patient satisfies one or more of the following conditions:
(g) the patient does not have a fasting plasma glucose of ≥7 mmol/L;
(h) the patient does not have T2D;
(i) the patient does not have HF;
(j) the patient does not have CKD stage 3 to 5; and/or
(k) the patient does not have severe hepatic impairment of Child-Pugh class 3.

In some embodiments of the methods disclosed herein, the patient accessed a webpage and provided answers to predetermined questions prior to the administration, and the patient was determined to be qualified to purchase the SGLT2 inhibitor based on the provided answers. In some embodiments, the administration does not require a medical prescription.

In some embodiments, the methods disclosed herein result in a risk reduction of developing microvascular and/or macrovascular complications. For example, at least in one embodiment, the methods disclosed herein result in a relative risk reduction of 28% for developing microvascular complications. In some embodiments, the methods disclosed herein reduce blood pressure. In some embodiments, the methods disclosed herein reduce body weight.

In the following description, certain details are set forth in order to provide a thorough understanding of various embodiments. However, one skilled in the art will understand that the disclosed embodiments may be practiced without these details. These and other embodiments will become apparent upon reference to the following detailed description and attached drawings.

### BRIEF DESCRIPTION OF THE DRAWINGS

FIG. 1 illustrates changes in insulin resistance, beta cell function, insulin, and glucose levels during the progression from prediabetes to T2D.

FIG. 2 illustrates predicted 24-hour glucosuria in patients with prediabetes in the DAPA-CKD (10 mg) and DAPA-HF (10 mg) studies, and for prediabetic patients taking 5 mg dapagliflozin and having various eGFR values.

FIG. 3 illustrates time to worsening in blood pressure, analyzing a subgroup of the DECLARE study. CI refers to Confidence interval; D refers to Dapa 10 mg; FAS refers to Full analysis set; HR refers to Hazard ratio; N refers to Number of participants; P refers to Placebo; refers to UACR Urinary albumin-to-creatinine ratio; and N at risk is the number of participants at risk at the beginning of the period.

FIG. 4 illustrates the study schema of the clinical trial described in Example 6.

FIG. 5 illustrates the Web App featuring a technology-assisted self-selection (TASS) tool with software-as-a-medical-device (SaMD) functions described in Example 7. DFL refers to drug facts label, AAD refers to ask a doctor, and SaMD refers to software-as-a-medical-device.

### DETAILED DESCRIPTION OF THE DISCLOSURE

The present disclosure relates to methods of reducing the risk of developing Type 2 diabetes in a patient in need thereof, the method comprising administering to the patient an effective amount of a sodium-glucose co-transporter 2 (SGLT2) inhibitor, e.g., dapagliflozin, wherein the patient has an HbA1c between 5.7% to 6.4% and/or a fasting glucose between 100 to 125 mg/dl.

The present disclosure also relates to methods of treating prediabetes in a patient in need thereof, the method comprising administering to the patient an effective amount of a SGLT2 inhibitor, e.g., dapagliflozin, wherein the patient has an HbA1c between 5.7% to 6.4% and/or a fasting glucose between 100 to 125 mg/dl.

In some embodiments, the SGLT2 inhibitor, e.g., dapagliflozin, or a pharmaceutically acceptable salt, solvate, mixed solvate, complex, or prodrug thereof, is administered at a dose of 2.5 mg, 5.0 mg, or 10 mg, once a day. In at least one embodiment, the dose is 2.5 mg. In at least one embodiment, the dose is 5 mg. In some embodiments, an SGLT2 inhibitor, such as dapagliflozin is in the form of a tablet.

In some embodiments, the SGLT2 inhibitor, e.g., dapagliflozin, is administered with at least one other therapeutic agent (such as, e.g., an antidiabetic agent) in the same or in a different composition, at the same time or at a different time.

### I. Definitions

The terms “comprising,” “having,” “including,” and “containing” are to be construed as open-ended terms (i.e., meaning “including, but not limited to,”) unless otherwise noted.

The term “or” is used herein to mean, and is used interchangeably with, the term “and/or,” unless context clearly indicates otherwise. The term “and/or” as used in a phrase such as “A and/or B” herein is intended to include the following embodiments: “A and B,” “A or B,” “A,” and “B.”

Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “treating” or “treatment” or “to treat” refer to therapeutic measures (e.g., administration of a medicament(s) to a subject) that cure, slow down, lessen symptoms of, and/or halt progression of a diagnosed pathologic disease, disorder, or condition, such as, e.g., prediabetes. Treatment need not result in a complete cure of the condition; partial inhibition or reduction of the condition being treated is encompassed by this term.

The phrase, “reducing the risk of developing” a pathologic condition and/or disorder, such as T2D, refers to preventing and/or slowing the development of the targeted pathologic condition and/or disorder, such as T2D. Thus, as used herein, “reducing the risk of developing T2D” includes reducing the incidence of developing T2D relative to a patient not being treated with the method disclosed herein.

The term “about” as used herein refers to within 20%, such as within 10%, and further such as within 5%, of a given value or range.

The term “other therapeutic agent” as used herein refers to a therapeutic agent other than the SLGT2 inhibitors of the present disclosure, or prodrugs thereof.

The term “prodrug” as used herein includes, for example, esters and carbonates that may be converted, for example, under physiological conditions or by solvolysis, to dapagliflozin. Thus, the term prodrug includes metabolic precursors of dapagliflozin that are pharmaceutically acceptable. The term prodrug also includes covalently bonded carriers that release dapagliflozin in vivo when such prodrug is administered to a patient. Non-limiting examples of prodrugs include esters and carbonates formed by reacting one or more hydroxyls of dapagliflozin with alkyl, alkoxy, or aryl substituted acylating agents employing procedures known to those skilled in the art to generate acetates, pivalates, methylcarbonates, benzoates, and the like. Various forms of prodrugs are known in the art. For examples of such prodrug derivatives, see: (1) Design of Prodrugs, edited by H. Bundgaard, (Elsevier, 1985) and Methods in Enzymology, Vol. 42, p. 309-396, edited by K. Widder, et al. (Academic Press, 1985); (2) A Textbook of Drug Design and Development, edited by Krogsgaard-Larsen and H. Bundgaard, Chapter 5 “Design and Application of Prodrugs,” by H. Bundgaard p. 113-191 (1991); (3) H. Bundgaard, Advanced Drug Delivery Reviews, 8, 1-38 (1992); (4) H. Bundgaard, et al., Journal of Pharmaceutical Sciences, 77, 285 (1988); and (5) N. Kakeya, et al., Chem Pharm Bull, 32, 692 (1984).

A “therapeutically effective amount” or “effective amount” refers to an amount of an active pharmaceutical ingredient that is effective to achieve a desired therapeutic result (e.g., of reducing the risk of developing Type 2 diabetes and/or treatment of prediabetes).

The terms “patient” and “subject” in need of reducing the risk of developing Type 2 diabetes (T2D) or treating prediabetes are used synonymously to refer to an adult human individual who has HbA1c values between 5.7% and 6.4% (5.7%≤HbA1c<6.5%) and/or fasting glucose between 100 to 125 mg/dl. (See American Diabetes Association. 2. Classification and diagnosis of diabetes: standards of medical care in diabetes-2021. Diabetes Care. 2021; 44 (Suppl 1): S15-33.) Also, as used herein “patients” or “subjects” in need of reducing the risk of developing T2D includes those prone to have T2D and those in whom T2D is to be prevented.

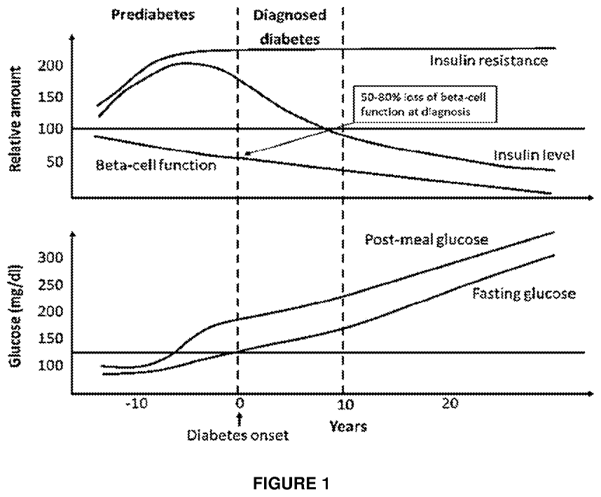

As shown in FIG. 1, disease progression from prediabetes to T2D starts with insulin resistance. Initially, the pancreas can cope with the increase in insulin demand, and it produces more insulin to overcome the insulin resistance. During this period, blood glucose levels remain in the normal range. After a period of time, the functionality of the beta cells that produce insulin in the pancreas start to decline and glucose levels will, as a consequence, increase. Initially, glucose levels increase into the prediabetes range, but over time with progressive beta cell decline, T2D glucose levels are reached. During this process, people have no noticeable symptoms, and they may already have developed complications by the time T2D is diagnosed. (Honigberg et al. Cardiovascular and kidney outcomes across the glycemic spectrum: insights from the UK biobank. J Amer Coll Cardiol. 2021; 78 (5): 453-64; Faghihimani et al. Evaluation of Peripheral Arterial Disease in Prediabetes. Int J Prev Med. 2014; 5 (9): 1099-105.)

The terms “administer,” “administering,” “administration,” and the like, as used herein, refer to methods that may be used to enable delivery of a drug, e.g., a SGLT2 inhibitor, as described herein. Administration techniques that can be employed with the agents and methods described herein can be found in e.g., Goodman and Gilman, The Pharmacological Basis of Therapeutics, current edition, Pergamon; and Remington's, Pharmaceutical Sciences, current edition, Mack Publishing Co., Easton, Pa. In at least one embodiment, the SGLT2 inhibitor is administered orally.

Administration of the SGLT2 inhibitor “in combination with one or more other therapeutic agents” includes simultaneous (concurrent) or consecutive administration, and, if simultaneous, in the same or different pharmaceutical composition (e.g., pill, tablet, capsule).

“Other therapeutic agents” include “standard of care heart failure (HF) medications,” “standard care Chronic kidney disease (CKD) agents,” and any other therapeutic agents as described herein.

“DAPA-HF,” “DAPA-CKD,” and/or “DECLARE” refer to AstraZeneca's placebo-controlled clinical studies. “DAPA-HF” refers to the DAPA-HF clinical study having Clinical Trial.gov number of NCT03036124. The “DAPA-HF” study is discussed in US PG Pub. No. 2021/0260083, which is incorporated by reference in its entirety. (See also Inzucchi S E, et al. Dapagliflozin and the incidence of type 2 diabetes in patients with heart failure and reduced ejection fraction: an exploratory analysis from DAPA-HF. Diabetes Care. 2021; 44 (2): 586-94.) “DAPA-CKD” refers to the DAPA-CKD clinical study having Clinical Trial.gov number of NCT03036150. The “DAPA-CKD” study is discussed in U.S. application Ser. Nos. 17/347,230 and 17/219,992, which are incorporated by reference in their entirety. (See also Rossing, et al. Dapagliflozin and New-Onset Type 2 Diabetes in Patients with Chronic Kidney Disease or Heart Failure: Pooled Analysis of the DAPA-CKD and DAPA-HF Trials. Lancet Diabetes Endocrinol. 2021; S2213-8587 (21) 00295-3.) “DECLARE” refers to the DECLARE-TIMI 58 clinical study having Clinical Trial.gov number of NCT01730534. (See Stephen et al. Dapagliflozin and Cardiovascular Outcomes in Type 2 Diabetes, N Engl J Med 2019; 380:347-357.)

“eGFR slope” is the change in eGFP over time. “eGFR” stands for the estimated glomerular filtration rate. The eGFR slope is a relevant measure of progressive loss of kidney function (microvascular complication) in prediabetes patients, and can be used to demonstrate clinical meaningfulness due to: 1) the prevalence of microvascular complications such as decline in kidney function in prediabetes patients prior to T2D diagnosis, 2) the higher risk of CKD in the prediabetic range compared to normoglycemia, and 3) the accelerated kidney function (eGFR) decline in patients close to the prediabetic state compared to normal age related decline.

### II. SGLT2 Inhibitors

As provided herein, SGLT2 inhibitors are used to reduce the risk of developing Type 2 diabetes or to treat prediabetes in a patient.

Sodium-glucose cotransporter 2 (SGLT2) is a sodium-dependent renal protein that is responsible for reabsorbing glucose back into the blood. SGLT2 inhibitors (also known as “Gliflozins”) are a class of medicine used to lower blood glucose in patients with type 2 diabetes by inhibiting renal SGLT2 proteins. As a result, more glucose is excreted in the urine.

In some embodiments, the SGLT2 inhibitor is chosen from those disclosed in U.S. Pat. No. 6,515,177, WO/2003/099836, U.S. PG Pub. No. 2006/0194809, U.S. PG Pub. No. 2006/0063722 A1, WO/2002/083066, U.S. PG Pub. No. 2003/0064935, U.S. Pat. No. 6,774,112, U.S. PG Pub. No. 2005/0209166, U.S. PG Pub. No. 2006/0074031, U.S. PG Pub. No. 2006/0035841, U.S. PG Pub. No. 2006/0009400, U.S. PG Pub. No. 2006/0025349, U.S. PG Pub. No. 2006/0122126, U.S. PG Pub. No. 2006/0019948, U.S. PG Pub. No. 2006/0194809, U.S. Pat. Nos. 6,908,905, 6,815,428, 6,555,519, 6,683,056, EP 598359 A1, JP 035988, U.S. Pat. No. 5,731,292, EP 0850948 A1, U.S. Pat. No. 6,048,842, JP 09188625 A, JP 09124685 A, JP 09124684, EP 773226 A1, U.S. Pat. No. 5,767,094, JP 08027006 A, EP 684254 A1, JP 10245391 (Dainippon), U.S. PG Pub. No. 2005/0233982 (Boehringer Ingelheim Corp.), U.S. PG Pub. No. 2005/0119192 (Kissei Pharmaceutical Co.), WO/2006/035796 (Kissei Pharmaceutical Co.), JP 2006/117651 (Taisho Pharmaceutical Co.), JP 2004/4359630 (Yamanouchi Pharmaceutical Co.), WO/2006/080421 (Chugai Seiyaku Kabushiki Kaishi), U.S. PG Pub. No. 2005/0233988 (Tanabe Seiyaku Co.), WO/2005/012321 (Tanabe Seiyaku Co.), U.S. Pat. No. 7,015,201 (Ajinomoto Co.), WO 2006/058597 (Merck Patent GmbH), WO 2006/011469 (Chugai Seiyaku Kabushiki Kaisha), U.S. PG Pub. No. 2003/0195235 (Johnson & Johnson), and WO 2006/037537 (Boehringer Ingelheim).

In some embodiments, the SGLT2 inhibitor is chosen from those disclosed in Tsujihara, K. et al., Chem. Pharm. Bull., 44:1174-1180 (1996); Hongu, M. et al., Chem. Pharm. Bull., 46:22-33 (1998); Hongu, M. et al., Chem. Pharm. Bull., 46:1545-1555 (1998); and Oku, A. et al., Diabetes, 48:1794-1800 (1999).

In some embodiments, the SGLT2 inhibitor may be dapagliflozin (FARXIGA®), canagliflozin (INVOKANA®), empagliflozin (JARDIANCE®), ertugliflozin (STEGLATRO®), sotagliflozin, ipragliflozin, tofogliflozin, or luseogliflozin, or a pharmaceutically acceptable salt, solvate, mixed solvate, complex, or prodrug of any of the foregoing.

In some embodiments, the SGLT2 inhibitor is dapagliflozin, or a pharmaceutically acceptable salt, solvate, mixed solvate, complex, or prodrug thereof, such as described in U.S. Pat. Nos. 6,414,126 and 6,515,117, which are incorporated by reference in their entireties.

Dapagliflozin (Forxiga™/Farxiga™) is a highly selective and reversible inhibitor of SGLT2. Dapagliflozin's mechanism of action results in a direct and insulin independent elimination of glucose by the kidneys, resulting in reduced blood glucose levels in type 2 diabetes (T2D) patients. In addition, dapagliflozin has a mild diuretic and natriuretic effect. The persistent loss of glucose, with associated calories in the urine, results in a consistent and maintained reduction of total body weight, predominantly a result of a reduction in fat mass including both visceral and subcutaneous adipose tissue. Moreover, dapagliflozin has also been shown to reduce BP and albuminuria, two prognostic risk factors for progression of CKD.

The chemical structure of dapagliflozin is:

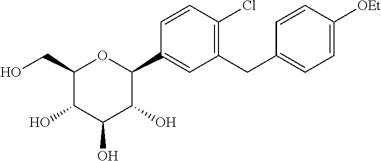 [SMILES: CCOc1ccc(Cc2cc([C@@H]3O[C@H](CO)[C@@H](O)[C@H](O)[C@H]3O)ccc2Cl)cc1]

In some embodiments, dapagliflozin is in the form of a non-crystalline solid. In some embodiments, dapagliflozin is in the form of a crystalline solid. In some embodiments, dapagliflozin is in the form of a(S)-propylene glycol ((S)-PG) solvate, which has the structure:

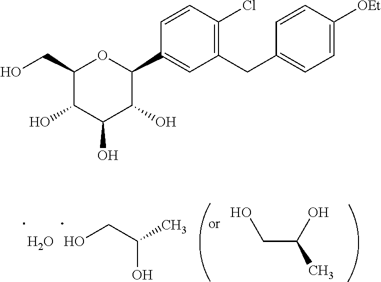

Methods for preparing a(S)-PG solvate of dapagliflozin, including a crystalline S-PG solvate, are provided in U.S. Pat. No. 7,919,598, the contents of which are incorporated by reference.

In some embodiments, the SGLT2 inhibitor, e.g., dapagliflozin, is administered with at least one other therapeutic agent.

### III. Other Therapeutic Agents

Administration of the SGLT2 inhibitor “in combination with one or more other therapeutic agents” includes simultaneous (concurrent) or consecutive administration, and, for simultaneous administration, in the same or different pharmaceutical composition (e.g., pill, tablet, capsule).

“Other therapeutic agents” include standard of care heart failure (HF) medications, standard of care Chronic kidney disease (CKD) agents, and any other therapeutic agents as described herein.

Exemplary standard of care CKD agents include angiotensin-converting enzyme inhibitors (ACE-Is or ACE inhibitors) and angiotensin receptor blockers (ARBs). Standard of care CKD agents and their dosages are well-known to medical practitioners who examine and treat patients with CKD. Representative examples of ACE inhibitors include captopril, enalapril, and lisinopril. Representative examples of ARBs include valsartan, losartan, and irbesartan.

Exemplary standard of care HF agents include, for example, medications or medication classes, other than SGLT2 inhibitors, that are used to treat HF, for instance, HFrEF. The standard of care HF agents, as described herein, may be used prior to and/or during administration of the SGLT2 inhibitor, e.g., dapagliflozin. Standard of care HF medications and their dosages are well-known to cardiologists and other medical practitioners who examine and treat patients with HFrEF. Exemplary standard of care HF agents include: angiotensin-converting enzyme (ACE) inhibitors; angiotensin receptor blockers (ARBs); beta blockers; mineralocorticoid receptor agents like mineralocorticoid receptor antagonists (MRA), and neprilysin inhibitors.

Other agents that may also be considered “standard of care HF agents,” include diuretics, and loop diuretics (e.g., furosemide, bumetanide, and torsemide), digoxin, heart pump medication, selective sinus node inhibitors, ivabradine (a sino-atrial (SA) node modulator), aldosterone antagonists, blood vessel dilators, calcium channel blockers (unless the patient has systolic heart failure), hydralazine/isosorbide dinitrate, or other HF medications within practice guidelines. (See Yancy C. W. et al., “ACC/AHA/HFSA focused update of the 2013 ACCF/AHA guideline for the management of heart failure: A report of the American College of Cardiology/American Heart Association task force on clinical practice guidelines and the Heart Failure Society of America, J Am Coll Cardiol. 70 (6): 776-803 (2017).)

Further disclosed herein are methods comprising administering to a patient in need thereof an effective amount of a SGLT2 inhibitor alone or in combination with at least one other therapeutic agent. In some embodiments, the other therapeutic agent is administered with the SGLT2 inhibitor in the same or in a different pharmaceutical composition, and, when in different pharmaceutical compositions, at the same or at a different time.

In some embodiments, the other therapeutic agent is an antidiabetic agent, anti-obesity agent, anti-hyperlipidemic agent, anti-atherosclerotic agent, anti-hypertensive agent, anti-platelet agent, antithrombotic agent, mineralocorticoid antagonist, diuretic, and/or anticoagulant agent. For example, in at least one embodiment, the other therapeutic agent is an antidiabetic agent such as a biguanide and/or a DPP4 inhibitor. An exemplary biguanide is metformin or a pharmaceutically acceptable salt thereof. Exemplary DPP4 inhibitors include saxagliptin, linagliptin, sitagliptin, and pharmaceutically acceptable salts thereof.

In some embodiments, the antidiabetic agent is chosen from biguanides. In some embodiments, the biguanide is metformin or pharmaceutically acceptable salts thereof. In some embodiments, the biguanide is metformin HCl. In some embodiments, the biguanide is phenformin.

In some embodiments, the antidiabetic agent is chosen from sulfonylureas and pharmaceutically acceptable salts thereof. In some embodiments, the sulfonylurea is chosen from glyburide, glimepiride, glipizide, gliclazide, and chlorpropamide. In some embodiments, the sulfonylurea is glyburide. In some embodiments, the sulfonylurea is glipizide.

In some embodiments, the antidiabetic agent is chosen from glucosidase inhibitors and pharmaceutically acceptable salts thereof. In some embodiments, the glucosidase inhibitor is chosen from acarbose and miglitol.

In some embodiments, the antidiabetic agent is chosen from PPAR y agonists. In some embodiments, the PPAR y agonist is chosen from thiazolidinediones. In some embodiments, the thiazolidinedione is chosen from troglitazone (e.g., Warner-Lambert's REZULIN®, disclosed in U.S. Pat. No. 4,572,912), rosiglitazone (e.g., as manufactured by SKB), pioglitazone (e.g., as manufactured by Takeda), Mitsubishi's MCC-555 (disclosed in U.S. Pat. No. 5,594,016), Glaxo-Wellcome's GL-262570, englitazone (e.g., CP-68722 manufactured by Pfizer), darglitazone (e.g., CP-86325 manufactured by Pfizer), isaglitazone (e.g., as manufactured by MIT/J&J), JTT-501 (JPNT/P&U), L-895645 (Merck), R-119702 (Sankyo/WL), N,N-2344 (Dr. Reddy/NN), or YM-440 (Yamanouchi).

In some embodiments, the thiazolidinedione is chosen from pioglitazone and rosiglitazone. In some embodiments, the thiazolidinedione is pioglitazone. In some embodiments, the thiazolidinedione is rosiglitazone.

In some embodiments, the antidiabetic agent is chosen from PPAR a/y dual agonists and pharmaceutically acceptable salts thereof. In some embodiments, the PPAR a/y dual agonist is chosen from AR-HO39242 (AstraZeneca), GW-409544 (Glaxo-Wellcome), KRP297 (Kyorin Merck), those disclosed by Murakami et al., “A Novel Insulin Sensitizer Acts As a Coligand for Peroxisome Proliferation-Activated Receptor Alpha (PPAR alpha) and PPAR gamma. Effect on PPAR alpha Activation on Abnormal Lipid Metabolism in Liver of Zucker Fatty Rats,” Diabetes, 47:1841-1847 (1998), and those disclosed in U.S. Pat. No. 6,414,002.

In some embodiments, the antidiabetic agent is chosen from aP2 inhibitors and pharmaceutically acceptable salts thereof. In some embodiments, the aP2 inhibitor is chosen from those disclosed in U.S. Pat. No. 6,548,529.

In some embodiments, the antidiabetic agent is chosen from DPP4 inhibitors and pharmaceutically acceptable salts thereof. In some embodiments, the DPP4 inhibitor is chosen from those disclosed in U.S. Pat. No. 6,395,767, WO 99/38501, WO 99/46272, WO 99/67279 (PROBIODRUG), WO 99/67278 (PROBIODRUG), WO 99/61431 (PROBIODRUG), NVP-DPP728A (1-[[[2-[(5-cyanopyridin-2-yl)amino]ethyl]amino]acetyl]-2-cyano-(S)-pyrrolidine) (Novartis), those disclosed by Hughes et al., Biochemistry, 38 (36): 11597-11603 (1999), TSL-225 (tryptophyl-1,2,3,4-tetrahydroisoquinoline-3-carboxylic acid (disclosed by Yamada et al., Bioorg. & Med. Chem. Lett., 8:1537-1540 (1998)), 2-cyanopyrrolidides, and 4-cyanopyrrolidides (as disclosed by Ashworth et al, Bioorg. & Med. Chem. Lett., 6 (22): 1163-1166 and 2745-2748 (1996)).

In some embodiments, the DPP4 inhibitor is chosen from saxagliptin, vildagliptin, linagliptin, alogliptin, and sitagliptin. In some embodiments, the DPP4 inhibitor is chosen from saxagliptin and pharmaceutically acceptable salts thereof. In some embodiments, the DPP4 inhibitor is saxagliptin. In some embodiments, the DPP4 inhibitor is saxagliptin HCl.

In some instances, the other therapeutic agent is an antidiabetic agent such as a biguanide (e.g., metformin) and/or a DPP4 inhibitor (e.g., saxagliptin, linagliptin, or sitagliptin). Representative examples of combination SGLT2 inhibitor+antidiabetic agent products include: dapagliflozin/metformin extended release (XIGDUO®), dapagliflozin/saxagliptin (QTERN®), dapagliflozin/saxagliptin/metformin (QTERNMET®), canagliflozin/metformin (INVOKAMET®), canagliflozin/metformin extended release (INVOKAMET XR®), empagliflozin/linagliptin (GLYXAMBI®), empagliflozin/metformin (SYNJARDY®), empagliflozin/metformin extended release (SYNJARDY XR®), ertugliflozin/metformin (STEGLUROMET®), and ertugliflozin/sitagliptin (STEGLUJAN®).

In some embodiments, the antidiabetic agent is chosen from meglitinides and pharmaceutically acceptable salts thereof. In some embodiments, the meglitinide is chosen from repaglinide, nateglinide (Novartis), and KAD1229 (PF/Kissei). In some embodiments, the meglitinide is repaglinide.

In some embodiments, the antidiabetic agent is chosen from glucokinase activators, DGAT-1 inhibitors, and pharmaceutically acceptable salts thereof. In some embodiments, the glucokinase activator is chosen from those disclosed in WO 2008/005964. In some embodiments, the DGAT-1 inhibitor is chosen from those disclosed in U.S. PG Pub No. 2008/0090876A1.

In some embodiments, the antidiabetic agent is chosen from insulin, GLP-1 receptor agonists, and pharmaceutically acceptable salts thereof. In some embodiments, the antidiabetic agent is insulin.

In some embodiments, the at least one other therapeutic agent is chosen from anti-obesity agents and pharmaceutically acceptable salts thereof. In some embodiments, the anti-obesity agent is chosen from beta 3 adrenergic agonists, lipase inhibitors, serotonin (and dopamine) reuptake inhibitors, thyroid receptor beta modulator, MCH-1 receptor antagonists, agonists of the 5-HT2c receptor, anorectic agents, Neuropeptide Y (NPY) antagonists, Leptin analogs, MC4 receptor agonists, and antagonists of the cannabinoid receptor.

In some embodiments, the beta 3 adrenergic agonist is chosen from AJ9677 (Takeda/Dainippon), SB-418790, L750355 (Merck), CP331648 (Pfizer), and other known beta 3 agonists as disclosed in U.S. Pat. Nos. 5,541,204, 5,770,615, 5,491,134, 5,776,983 and 5,488,064. In some embodiments, the beta 3 adrenergic agonist is chosen from AJ9677, L750355, and CP331648.

In some embodiments, the at least one other therapeutic agent is chosen from anti-hyperlipidemic agents and pharmaceutically acceptable salts thereof. In some embodiments, the hyperlipidemic agent is chosen from HMG CoA reductase inhibitors. In some embodiments, the HMG-COA reductase inhibitor is chosen from mevastatin and related compounds as disclosed in U.S. Pat. No. 3,983,140, lovastatin (mevinolin) and related compounds as disclosed in U.S. Pat. No. 4,231,938, pravastatin and related compounds as disclosed in U.S. Pat. No. 4,346,227, simvastatin and related compounds as disclosed in U.S. Pat. Nos. 4,448,784 and 4,450,171, and rosuvastatin and related statin compounds as disclosed in U.S. Pat. No. 5,753,675.

In some embodiments, the at least one other therapeutic agent is chosen from anti-hypertensive agents and pharmaceutically acceptable salts thereof. In some embodiments, the anti-hypertensive agent is chosen from beta adrenergic blockers, calcium channel blockers (L-type and/or T-type), diuretics, renin inhibitors, ACE inhibitors, AT-1 receptor antagonists, ET receptor antagonists as disclosed in U.S. Pat. Nos. 5,612,359 and 6,043,265, Dual ET/AII antagonists as disclosed in WO 00/01389, neutral endopeptidase (NEP) inhibitors, vasopepsidase inhibitors, and nitrates.

In some embodiments, the anti-hypertensive agent is chosen from bisoprolol, carvedilol, metaprolol succinate, diltiazem, verapamil, nifedipine, amlodipine, mibefradil, chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone, torsemide, indapamide, metolazone, triamterene, eplerenone, captopril, zofenopril, fosinopril, enalapril, ceranopril, cilazopril, delapril, pentopril, quinapril, ramipril, lisinopril, perindopril, trandolapril, losartan, irbesartan, valsartan, candesartan, sitaxsentan, atrsentan, omapatrilat, gemopatrilat, hydralazine, isosorbide dinitrate, nitroglycerin, and nitroprusside.

In some embodiments, the at least one other therapeutic agent is chosen from anti-platelet agents and pharmaceutically acceptable salts thereof. In some embodiments, the anti-platelet agent is chosen from clopidogrel, ticlopidine, prasugrel, and aspirin.

In some embodiments, the at least one other therapeutic agent is chosen from antithrombotic agents, anticoagulant agents, and pharmaceutically acceptable salts thereof. In some embodiments, the antithrombotic agent and/or anticoagulant agent is chosen from thrombin inhibitors, platelet aggregation inhibitors, PAI-1 inhibitors, inhibitors of α-2-antiplasmin, thromboxane receptor antagonists, prostacyclin mimetics, and phosphodiesterase (PDE) inhibitors.

In some embodiments, the antithrombotic agent and/or anticoagulant agent is chosen from clopidogrel, ticlopidine, prasugrel (Eli Lilly), XR-330, T-686, anti-α-2-antiplasmin antibody, ifetroban, dipyridamole, cilostazol, aspirin, ifetroban, picotamide, and ketanserin.

In some embodiments, the SGLT2 inhibitor, e.g., dapagliflozin, is administered with at least one other therapeutic agent in the same or in a different composition, and if in different compositions, at the same or at a different time. In some embodiments, the at least one other therapeutic agent is administered before, after, or concurrently with the SGLT2 inhibitor, e.g., dapagliflozin.

### IV. Methods of Reducing the Risk of Developing Type 2 Diabetes or Treating Prediabetes

The present disclosure relates to methods of reducing the risk of developing Type 2 diabetes in a patient in need thereof, the method comprising administering to the patient, an effective amount of a sodium-glucose co-transporter 2 (SGLT2) inhibitor, e.g., dapagliflozin wherein the patient has an HbA1c between 5.7% to 6.4% and/or a fasting glucose between 100 to 125 mg/dl.

The present disclosure also relates to methods of treating prediabetes in a patient in need thereof, the method comprising administering to the patient, an effective amount of a SGLT2 inhibitor, e.g., dapagliflozin, wherein the patient has an HbA1c between 5.7% to 6.4% and/or a fasting glucose between 100 to 125 mg/dl.

In some embodiments, the patient does not have Type 1 diabetes (T1D) or Type 2 diabetes (T2D). In some embodiments, the patient was not previously administered a prescription medicine for diabetes. In some embodiments, the patient does not have chronic kidney disease (CKD) and/or heart failure (HF).

In at least one embodiment, the SGLT2 inhibitor is dapagliflozin, or a pharmaceutically acceptable salt, solvate, mixed solvate, complex, or prodrug. In some embodiments, dapagliflozin is in the form of a non-crystalline solid. In some embodiments, dapagliflozin is in the form of a crystalline solid. In some embodiments, dapagliflozin is in the form of a(S)-propylene glycol ((S)-PG) solvate, which has the structure:

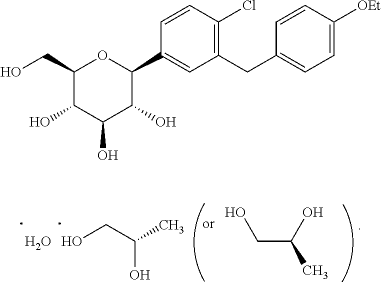

An effective amount or therapeutically effective amount refers to an amount of at least one compound of the present disclosure or a pharmaceutical composition comprising at least one such compound of the present disclosure that, when administered to a patient, either as a single dose or as part of a series of doses, is effective to produce at least one therapeutic effect. The dose may depend upon the body mass, weight, and/or blood volume of the patient. Patients may generally be monitored for therapeutic effectiveness using assays suitable for the disease, disorder, and/or condition being treated or prevented. The level of a compound that is administered to a patient may be monitored by determining the level of the compound (or a metabolite of the compound) in a biological fluid, for example, in the blood, blood fraction (e.g., serum), urine, and/or other biological sample from the patient. Any method practiced in the art to detect the compound, or metabolite thereof, may be used to measure the level of the compound during the course of a therapeutic regimen.

The dose of a compound described herein may depend upon the patient's condition, that is, stage of the disease, severity of symptoms caused by the disease, general health status, as well as age, gender, and weight, and other factors apparent to a person of ordinary skill in the medical art.

In some embodiments, the at least one compound chosen from SGLT2 inhibitors, e.g., dapagliflozin, and prodrugs thereof is administered at a dose equivalent of from about 1 to about 500 mg/day dapagliflozin. In some embodiments, the at least one compound chosen from SGLT2 inhibitors, e.g., dapagliflozin, and prodrugs thereof is administered at a dose equivalent of from about 2 to about 400 mg/day dapagliflozin. In some embodiments, the at least one compound chosen from SGLT2 inhibitors, e.g., dapagliflozin, and prodrugs thereof is administered at a dose equivalent of from about 0.5 to about 200 mg/day dapagliflozin. In some embodiments, the at least one compound chosen from SGLT2 inhibitors, e.g., dapagliflozin, and prodrugs thereof is administered at a dose equivalent of from about 1 to about 100 mg/day dapagliflozin. In some embodiments, the at least one compound chosen from SGLT2 inhibitors, e.g., dapagliflozin, and prodrugs thereof is administered at a dose equivalent of from about 1 to about 50 mg/day dapagliflozin. In some embodiments, the at least one compound chosen from SGLT2 inhibitors, e.g., dapagliflozin, and prodrugs thereof is administered at a dose equivalent of from about 1 to about 20 mg/day dapagliflozin. In some embodiments, the at least one compound chosen from SGLT2 inhibitors, e.g., dapagliflozin, and prodrugs thereof is administered at a dose equivalent of from about 2.5 to about 20 mg/day dapagliflozin. In some embodiments, the at least one compound chosen from SGLT2 inhibitors, e.g., dapagliflozin, and prodrugs thereof is administered at a dose equivalent of from about 2.5 to about 10 mg/day dapagliflozin. In some embodiments, the at least one compound chosen from SGLT2 inhibitors, e.g., dapagliflozin, prodrugs thereof is administered at a dose equivalent of about 10 mg/day dapagliflozin. In some embodiments, the at least one compound chosen from SGLT2 inhibitors, e.g., dapagliflozin, and prodrugs thereof is administered at a dose equivalent of about 5 mg/day dapagliflozin n. In some embodiments, the at least one compound chosen from SGLT2 inhibitors, e.g., dapagliflozin, and prodrugs thereof is administered at a dose equivalent of about 2.5 mg/day dapagliflozin.

In some embodiments, the SGLT2 inhibitor (e.g., dapagliflozin) is administered orally to the patient, one time a day. In some embodiments, dapagliflozin is administered orally to the patient at a dose of 2.5 mg, 5.0 mg, or 10 mg, once a day. In at least one embodiment, the oral dose of dapagliflozin administered is 2.5 mg. In at least one embodiment, the oral dose of dapagliflozin administered is 5.0 mg.

In some embodiments, the method further comprises administering at least one other therapeutic agent to the patient. In some embodiments, the other therapeutic agent is administered with the SGLT2 inhibitor in the same composition. In some embodiments, the other therapeutic agent is administered with the SGLT2 inhibitor in different pharmaceutical compositions, and at the same or different time. In some embodiments, the weight ratio for the combination of the at least one compound chosen from SGLT2 inhibitors, e.g., dapagliflozin, and prodrugs thereof and the at least one other therapeutic agent is within the range of from about 0.01:1 to about 300:1. In some embodiments, the weight ratio for the combination of the at least one compound chosen from SGLT2 inhibitors, e.g., dapagliflozin, and prodrugs thereof and the at least one other therapeutic agent is within the range of from about 0.1:1 to about 200:1. In some embodiments, the weight ratio for the combination of the at least one compound chosen from SGLT2 inhibitors, e.g., dapagliflozin, and prodrugs thereof and the at least one other therapeutic agent is within the range of from about 0.2:1 to about 100:1.

In some embodiments, the other therapeutic agent is an antidiabetic agent, anti-obesity agent, anti-hyperlipidemic agent, anti-atherosclerotic agent, anti-hypertensive agent, anti-platelet agent, antithrombotic agent, or anticoagulant agent. In some embodiments, the other therapeutic agent is an antidiabetic agent. In some embodiments, the antidiabetic agent is a biguanide and/or a DPP4 inhibitor. In some embodiments, the biguanide is metformin or a pharmaceutically acceptable salt thereof. In some embodiments, the DPP4 inhibitor is saxagliptin, linagliptin, or sitagliptin, or a pharmaceutically acceptable salt thereof.

In some embodiments, the at least one other therapeutic agent is an angiotensin-converting enzyme inhibitor (ACE inhibitor), such as captopril, enalapril, and lisinopril. In some embodiments, the at least one other therapeutic agent is an angiotensin receptor blocker (ARB), such as valsartan, losartan, and irbesartan.

In some embodiments, prior to the administration, the patient had an eGFR of ≥45 and ≤90 mL/min/1.73 m². In some embodiments, prior to the administration, the patient had an eGFR of ≥60 and ≤90 mL/min/1.73 m². In some embodiments, prior to the administration, the patient had an eGFR of ≥39 and ≤67 mL/min/1.73 m². For example, in some embodiments, prior to the administration, the patient had an eGFR of ≥60 mL/min/1.73 m². In some embodiments, prior to the administration, the patient had an eGFR of eGFR of ≥60 mL/min/1.73 m² and ≤65 mL/min/1.73 m². For example, prior to the administration, the patient had an eGFR of eGFR of 61, 62, 63, 64, 65 and/or 66 mL/min/1.73 m².

In some embodiments, the method reduces the risk of developing T2D in the patient. In some embodiments, the method reduces the risk of developing T2D relative to a patient not being treated with an effective amount of an SGLT2 inhibitor. In some embodiments, reducing the risk of developing T2D is assessed by measuring the incidence of developing T2D relative to a patient not being treated with an effective amount of an SGLT2 inhibitor. In some embodiments, the risk of developing T2D is measured relative to a baseline value measured for the patient prior to treatment with an effective amount of an SGLT2 inhibitor. In some embodiments, the risk of developing T2D is measured relative to one or more patients who is not being treated with an effective amount of an SGLT2 inhibitor.

In some embodiments, the method results in a relative risk reduction of 25% or more for developing T2D in the patient. For example, in some embodiments the method results in a relative risk reduction of 25%, 26%, 27%, 28%, 29%, 30%, 31%, 32%, 33%, 34%, 35%, 36%, 37%, 38%, 39%, and/or 40%. In some embodiments, the method results in a relative risk reduction of 30% for developing T2D. In some embodiments, the method results in a relative risk reduction from 25% to 35%, from 25% to 30%, from 28% to 32%, and/or from 29% to 31% for developing T2D. In some embodiments, the relative risk reduction for developing T2D is measured relative to a baseline value measured for the patient prior to treatment with an effective amount of an SGLT2 inhibitor. In some embodiments described herein, the relative reduction for developing T2D is measured relative to one or more patients who is not being treated with an effective amount of an SGLT2 inhibitor.

In some embodiments, the method results in an absolute risk reduction of 3% or more for developing T2D during a period of 1.75 years. For example, in at least one embodiment, the method results in an absolute risk reduction of 3.0%, 3.5%, 4.0%, 4.5%, 5.0%, 5.5%, 6.0%, 6.5%, 7.0%, 7.5%, 8.0%, 8.5%, 9.0%, 9.5% and/or 10.0% or more for developing T2D during a period of 1.75 years. In some embodiments, the absolute risk reduction for developing T2D is measured relative to a baseline value measured for the patient prior to treatment with an effective amount of an SGLT2 inhibitor. In some embodiments, the absolute risk reduction for developing T2D is measured relative to one or more patients who is not being treated with an effective amount of an SGLT2 inhibitor.

In some embodiments, the method results in a hazard ratio from 0.65 to 0.8. For example, in some embodiments the method results in a hazard ratio of 0.65, 0.66, 0.67, 0.68, 0.69, 0.70, 0.71, 0.72, 0.73, 0.74, 0.75, 0.76, 0.77, 0.78, 0.79 and/or 0.80. For example, in some embodiments, the hazard ratio is 0.69. In some embodiments, the hazard ratio is 0.72. In some embodiments, the hazard ratio is 0.75.

In some embodiments, the administration reduces the risk of developing microvascular and/or macrovascular complications in the patient. For example, in some embodiments, the administration results in a relative risk reduction of 25%, 26%, 27%, 28%, 29%, 30%, 31%, 32%, 33%, 34% and/or 35% for developing microvascular complications. In some embodiments, the administration reduces the risk of developing microvascular and/or macrovascular complications relative to a patient not being treated with an effective amount of an SGLT2 inhibitor. In some embodiments, the risk of developing microvascular and/or macrovascular complications is measured relative to a baseline value measured for the patient prior to treatment with an effective amount of an SGLT2 inhibitor. In some embodiments, the risk of developing microvascular and/or macrovascular complications is measured relative to one or more patients who is not being treated with an effective amount of an SGLT2 inhibitor.

In some embodiments, the administration reduces blood pressure in the patient. In some embodiments, the administration reduces blood pressure relative to a patient not being treated with an effective amount of an SGLT2 inhibitor.

In some embodiments, the administration reduces body weight. In some embodiments, the administration reduces body weight relative to a patient not being treated with an effective amount of an SGLT2 inhibitor.

In some embodiments, the patient being treated satisfies one or more of the following conditions:
(a) the patient has a BMI of ≥30 kg/m²;
(b) the patient has a first degree relative with T2D;
(c) the patient has a medical history of hypertension;
(d) the patient has a medical history of dyslipidemia;
(e) the patient has prior gestational diabetes; and/or
(f) the patient has polycystic ovary syndrome.

In some embodiments, the patient satisfies one or more of the conditions (a) to (f) listed above. In some embodiments, the patient satisfies each of the conditions (a) to (f) listed above.

In some embodiments of the disclosed methods herein, the patient has a BMI of ≥25 kg/m². For example, in some embodiments, the patient has a BMI of ≥30 kg/m².

In some embodiments, the patient is ≥45 years old and has a body mass index of ≥30 kg/m² for non-Asians or >27 kg/m² for Asians.

In some embodiments, the patient being treated satisfies one or more of the following conditions:
(a) the patient does not have a fasting plasma glucose of ≥7 mmol/L;
(b) the patient does not have T2D;
(c) the patient does not have HF;
(d) the patient does not have CKD stage 3 to 5; and/or
(e) the patient does not have severe hepatic impairment of Child-Pugh class 3.

In some embodiments, the patient satisfies one or more of the conditions (a) to (e) listed above. In some embodiments, the patient satisfies each of the conditions (a) to (e) listed above.

In some embodiments, prior to the administration, the patient accessed a webpage and provided answers to predetermined questions; and the patient was determined to be qualified to purchase the SGLT2 inhibitor based on the provided answers. In some embodiments, the patient receives a medical prescription after being deemed qualified to purchase the SGLT2 inhibitor. In some embodiments, the administration does not require a medical prescription.

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Examples

### Example 1: Analyses of DAPA-HF, DAPA-CKD, and DECLARE Subpopulations

DAPA-HF primarily studied dapagliflozin in reducing the risk of composite CV death and HF in a broad patient population with HFrEF irrespective of T2D status. A total of 2605 of 4744 patients did not have T2D at baseline. DAPA-CKD primarily studied dapagliflozin in reducing the risks of major adverse kidney and CV events and all-cause mortality in patients with CKD irrespective of T2D status. A total of 1398 of 4304 patients did not have T2D at baseline.

As shown in Table 1, exploratory analyses of the DAPA-HF and DAPA-CKD studies demonstrated that treatment with dapagliflozin reduced the incidence of developing T2D in non-diabetic patients with HF and CKD, respectively. Furthermore, a meta-analysis of the DAPA-HF and DAPA-CKD population also demonstrated that treatment with dapagliflozin reduced the incidence of developing T2D.

TABLE 1
New Onset T2D in Non-diabetic Populations in
DAPA-HF and DAPA-CKD Studies

| | | Event rate | |
|---|---|---|---|
| | Total | (per 100 | |
| | Number of | patient-years of | |
| Study | participants | follow-up) | HR (95% CI) |
| DAPA-HF | 2605 | Dapa: 5.0 | 0.68 (0.50, 0.94) |
| HbA1C < 6.5%^{a} | | Placebo: 3.4 | |
| Pre-diabetic 5.7% ≤ | 1748 | Dapa: 5.3 | 0.72 (0.52, 0.99) |
| HbA1C < 6.5 | | Placebo: 7.5 | |
| DAPA-CKD | 1398 | Dapa: 1.4 | 0.62 (0.36, 1.07) |
| HbA1C < 6.5%^{a} | | Placebo: 2.3 | |
| Pre-diabetic 5.7% ≤ | 660 | Dapa: 2.7 | 0.59 (0.33, 1.04) |
| HbAlc < 6.5 | | Placebo: 4.4 | |
| Meta-analysis DAPA- | 4003 | Dapa: 2.6 | 0.67 (0.51, 0.88) |
| HF/CKD | | Placebo: 3.9 | |
| HbA1c < 6.5%^{b} | | | |
| Pre-diabetic 5.7% ≤ | 2408 | Dapa: 4.2 | 0.69 (0.52, 0.91) |
| HbAlc < 6.5^{b} | | Placebo: 6.2 | |

CI Confidence interval; Dapa dapagliflozin; HBa1c Glycated Haemoglobin; HR Hazard ratio; T2D Type 2 Diabetes Mellitus
Source:
^{a}results published (Inzucci et al 2001)
^{b}results published (Rossing et al 2001)
Exploratory analyses for DAPA-HF (iemt5381 outputs)
Exploratory analyses for DAPA-CKD (iemt4391 outputs)

The DECLARE study enrolled a broad T2D patient population. By using parameters to select for a relatively healthy population that is close to the prediabetes population, the DECLARE population was split into 4 subgroups. The four subgroups in DECLARE are those with eGFR ≥60 ml/min/1.73 m², no history of HF at baseline and:
1. HbA1c <7%, SBP <140 mmHg, and UACR ≤300 mg/g, or
2. HbA1c <7%, SBP <140 mmHg, and UACR <30 mg/g, or
3. T2D <5 years and UACR ≤300 mg/g, or
4. T2D <5 years and UACR ≤30 mg/g.

In DECLARE, the placebo eGFR slopes in the subgroups close to prediabetes are less steep than that of the total population in DECLARE (−1.51 to −2.36 ml/min/1.73 m²/yr vs −2.17 to −2.55 ml/min/1.73 m²/yr) as shown in Table 2, but the decline in eGFR is still faster than that in the average population of 1.00 ml/min/1.73 m²/yr. (See Baba et al. Longitudinal Study of the Decline in Renal Function in Healthy Subjects. PLOS One. 2015; 10 (6).) A steeper slope in people with IGT (mean baseline HbA1c of around 5.8%) was also seen, which is in line with the DECLARE subgroups. (See Currie et al. Effect of valsartan on kidney outcomes in people with impaired glucose tolerance. Diabetes Obes Metab. 2017; 19 (6): 791-99.) Furthermore, the higher event rates of people with prediabetes compared with normoglycemia reaching eGFR <60 ml/min/1.73 m² support an accelerated eGFR decline. (See Chen et al. Association between Prediabetes and Renal Dysfunction from a Community-based Prospective Study. Int J Med Sci. 2020; 17 (11): 1515-21; Li et al. Risk of chronic kidney disease defined by decreased estimated glomerular filtration rate in individuals with different prediabetic phenotypes. BMJ Open Diabetes Res Care. 2020; 8 (1).) Together, the subpopulations close to prediabetes in DECLARE may be a good reference indicative of prediabetes patients.

The treatment effect of dapagliflozin on chronic slope in DECLARE subpopulations close to prediabetes and the prediabetes populations of DAFA-HF and DAPA-CKD are summarized in Table 2. As shown in Table 2, the treatment effect is larger in patients with a faster decline in eGFR. In the DECLARE populations closest to prediabetes, the decline in eGFR is at a magnitude that is seen as clinically relevant since it is associated with an HR for subsequent end stage kidney disease (ESKD) of ˜0.7. (See Inker et al., GFR Slope as a Surrogate End Point for Kidney Disease Progression in Clinical Trials: A Meta-Analysis of Treatment Effects of Randomized Controlled Trials. J Am Soc Nephrol. 2019; 30 (9): 1735-45; Levey et al., GFR decline as an end point for clinical trials in CKD. Am J Kidney Dis. 2014; 64 (6): 821-35.)

In the subpopulation with diabetes duration <5 years and UACR <30 mg/g, dapagliflozin slowed the rate of eGFR decline by 1.03 and 0.96 ml/min/1.73 m²/yr at 2 and 3 year, respectively. In the subpopulation with HbA1c <7%, SBP <140 mmHg, and UACR <30 mg/g, dapagliflozin slowed the rate of eGFR decline by 1.25 and 1.17 ml/min/1.73 m²/yr at 2 and 3 year, respectively.

Together, eGFR slope is a relevant measure of progressive loss of kidney function (microvascular complication) in prediabetes patients to demonstrate clinically meaningfulness due to: 1) the prevalence of microvascular complications such as decline in kidney function in prediabetes patients prior to T2D diagnosis, 2) the higher risk of CKD in the prediabetic range compared to normoglycemia, and 3) the accelerated kidney function (eGFR) decline in patients close to the prediabetic state compared to normal age-related decline. The risks of CKD and accelerated eGFR decline may contribute to the development of CKD, as defined by eGFR <60 ml/min/1.73 m², which in turn increases the risk of ESKD 5- to 20-fold. (See Gansevoort et al., Lower estimated GFR and higher albuminuria are associated with adverse kidney outcomes. A collaborative meta-analysis of general and high-risk population cohorts. Kidney Int. 2011; 80 (1): 93-104.)

The results from the dapagliflozin exploratory analyses in Table 2 support the benefits of dapagliflozin in reducing eGFR decline in prediabetes patients. Therefore, being able to measure the progression of kidney function decline or progression to CKD is informative and clinical benefit lies in normalizing the rate of eGFR decline in these patients. Additionally, early identification and treatment are more effective than later intervention for the prevention of adverse renal outcomes. (See Schievink et al., Early renin-angiotensin system intervention is more beneficial than late intervention in delaying end-stage renal disease in patients with type 2 diabetes. Diabetes Obes Metab. 2016; 18 (1): 64-71.) Dapagliflozin treatment in the prediabetic state may significantly impact the course of kidney function decline in these at-risk patients. Therefore, a reduction in eGFR slope is a relevant method to demonstrate the reduction of microvascular complications as a relevant clinical outcome in prediabetes patients.

TABLE 2
eGFR chronic slope differences in DAPA-HF, DAPA-CKD, and DECLARE

| | | | | | | | Difference between | | | |
|---|---|---|---|---|---|---|---|---|---|---|
| | | | Dapa 10 mg | | Placebo | | Dapa 10 mg and Placebo | | | |
| Population | Slope at | N | Slope (SE) | N | Slope (SE) | | Slope diff | SE | 95% CI | P-value |
| DAPA-HF (prediabetes) | 14-720 days | 860 | −0.92 (0.27) | 888 | −2.27 | (0.26) | 1.35 | 0.38 | (0.61, 2.09) | 0.0004 |
| DAPA-CKD (prediabetes) | 14 days-30 months | 2152 | −1.69 (0.22) | 2152 | −3 13 | (0 22) | 1.44 | 0.32 | (0.82, 2.05) | <.0001 |
| DECLARE (Total | 6 months-2 yrs | 7439 | −0.83 (0.08) | 7242 | −2.17 | (0.08) | 1.34 | 0.11 | (0.13, 1.55) | <0.001 |
| Population) | 6 months-3 yrs | 7034 | −1.18 (0.05) | 6736 | −2.36 | (0.05) | 1.18 | 0.07 | (1.04, 1.32) | <0.001 |
| | 6 months-4 yrs | 5985 | −1.54 (0.04) | 5707 | −2.55 | (0.04) | 1.01 | 0.06 | (0.90, 1.12) | <0.001 |
| DECLARE^{a} (HbAlc <7%, | 6 months-2 yrs | 351 | −0.24 (0.33) | 344 | −181 | (0.34) | 1.56 | 0.47 | (0.64, 2.49) | <0.001 |
| SBP <140 mmHg, and | 6 months-3 yrs | 332 | −0.51 (0.21) | 324 | −2 36 | (0 21) | 1.85 | 0.30 | (1.27, 2.44) | <0.001 |
| UACR ≤300 mg/g) | 6 months-4 yrs | 305 | −0.92 (0.16) | 299 | −1 99 | (0.16) | 1.08 | 0.23 | (0.62, 1 53) | <0.001 |
| DECLARE^{a} (HbAlc <7%, | 6 months-2 yrs | 305 | −0.26 (0.33) | 282 | −1.51 | (0.34) | 1.25 | 0.47 | (0.32, 2.19) | 0.008 |
| SBP <140 mmHg, and | 6 months-3 yrs | 287 | −0.56 (0.22) | 264 | −2.28 | (0.22) | 1.71 | 0.31 | (1.10, 2 32) | <0.001 |
| UACR <30 mg/g) | 6 months-4 yrs | 264 | −0.92 (0.16) | 247 | −1.90 | (0.16) | 0.98 | 0.23 | (0.53, 1.43) | <0.001 |
| DECLARE^{a} (Diabetes | 6 months-2 yrs | 1323 | −0.77 (0.17) | 1351 | −1.65 | (0.17) | 0.88 | 0.24 | (0.42, 1.34) | <0.001 |
| duration <5 years, | 6 months-3 yrs | 1257 | −1.11 (0.11) | 1263 | −2.02 | (0.11) | 0.92 | 0.16 | (0.61, 1.23) | <0.001 |
| UACR ≤300 mg/g) | 6 months-4 yrs | 1070 | −1.42 (0.09) | 1069 | −2.28 | (0.09) | 0.86 | 0.13 | (0.62, 1.11) | <0.001 |
| DECLARE^{a} (Diabetes | 6 months-2 yrs | 1039 | −0.56 (0.19) | 1107 | −1.59 | (0.18) | 1.03 | 0.26 | (0.53, 1.54) | <0.001 |
| duration <5 years, | 6 months-3 yrs | 995 | −0.92 (0.12) | 1042 | −1.88 | (0.12) | 0.96 | 0.17 | (0.62, 1.29) | <0.001 |
| UACR ≤30 mg/g) | 6 months-4 yrs | 853 | −1.27 (0.10) | 895 | −2.13 | (0.09) | 0.86 | 0.13 | (0.60, 1.12) | <0.001 |

^{a}DECLARE subgroups also had eGFR ≥60 ml/min/1.73 m² and no history of HF at baseline.
eGFR slope calculated in DAPA-HF, DAPA-CKD, and DECLARE is consistent with how they were calculated in the respective studies

### Example 2: Microvascular and Macrovascular Clinical Benefit of Dapagliflozin in Prediabetes Patients

Dapagliflozin reduced the risk for kidney events in patients with T2D, HF, and CKD independent of glycemic state. In DAPA-CKD, dapagliflozin reduced the risk of the primary composite outcome of worsening of kidney function or death in patients with diabetes (HR: 0.64; 95% CI 0.52 to 0.79) and without diabetes (HR: 0.50; 95% CI 0.35 to 0.72) (p value for interaction=0.24). The patients without diabetes (or T2D) included those with prediabetes. In patients with and without T2D, including those with prediabetes, dapagliflozin treatment slowed the decline in eGFR slope. T2D patients had a placebo-corrected difference of 1.18 ml/min/1.73 m²/yr (95% CI 0.79 to 1.56; p<0.0001) and patients without T2D had a placebo-corrected difference of 0.46 ml/min/1.73 m²/yr (95% CI-0.10 to 1.03; p=0.11) from baseline to end of treatment. T2D patients had a placebo-corrected difference of 2.26 ml/min/1.73 m²/yr (95% CI 1.88 to 2.64) and patients without T2D had a placebo corrected difference of 1.29 ml/min/1.73 m²/yr (95% CI 0.73 to 1.85) in slope from two weeks to end of treatment.

Further, in patients without CKD, dapagliflozin also reduced the rate of decline in kidney function. (See Jhund et al., Efficacy of Dapagliflozin on Renal Function and Outcomes in Patients With Heart Failure With Reduced Ejection Fraction: Results of DAPA-HF. Circulation. 2021; 143 (4): 298-309; Mosenzon et al. Effects of dapagliflozin on development and progression of kidney disease in patients with type 2 diabetes. Lancet Diabetes Endocrinol. 2019; 7 (8): 606-617; Wheeler et al. Effects of dapagliflozin on major adverse kidney and cardiovascular events in patients with diabetic and non-diabetic chronic kidney disease. Lancet Diabetes Endocrinol. 2021; 9 (1): 22-31.) These findings suggest that treatment with dapagliflozin could also be useful for early prevention of CKD. Indeed, data from DECLARE demonstrated that T2D patients with eGFR in the range of 60 to 90 and ≥90 ml/min/1.73 m² experienced renal benefits following dapagliflozin treatment in terms of reduced eGFR decline and albuminuria.

Predefined subgroup analyses of the primary and secondary endpoints in DAPA-HF and DAPA-CKD demonstrated that dapagliflozin was effective at reducing CV morbidity and mortality and the risk of major adverse kidney events independent of diabetes status. In DAPA-HF, dapagliflozin reduced the risk of the primary composite outcome (CV death, hospitalization for HF, or urgent HF visit) in patients with diabetes (HR: 0.75; 95% CI 0.63 to 0.90) and without diabetes (HR: 0.73; 95% CI 0.60 to 0.88) (p value for interaction=0.80). Taken together, these results support the benefits of dapagliflozin in reducing the risks of CV and major adverse kidney events in people without diabetes, which includes the prediabetes patients.

### Example 3: Dapagliflozin Reduces Blood Pressure

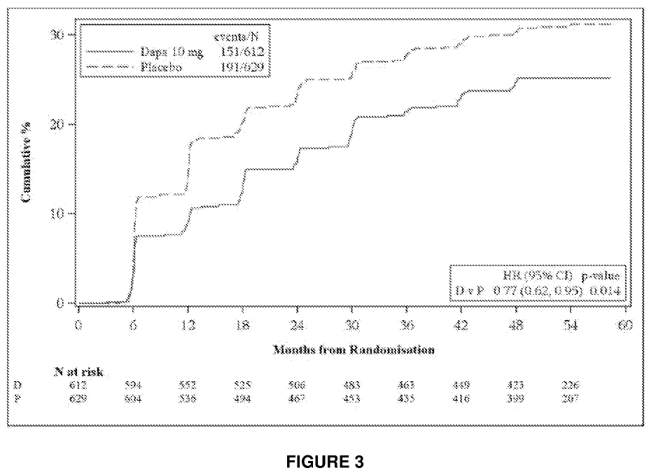

An exploratory subgroup analysis was performed to determine the effect of dapagliflozin using available data in the DECLARE subgroup that is closer to prediabetes patients. As illustrated in FIG. 3, treatment with dapagliflozin lowered the risk of worsening blood pressure compared with placebo (in the time to worsening in blood pressure category).

FIG. 3 is an analysis of participants with the following criteria: HbA1c at baseline <7%, eGFR at baseline ≥60 ml/min/1.73 m², no history of HF at baseline, and UACR≤300 mg/g. For the purposes of the analysis, 1 month corresponds to 30 days. FIG. 3 analyzed the time from randomization to first occurrence of event or censoring and a two-sided p value is displayed. HR, CI, and p-value are from a Cox proportional hazard model. Blood pressure categories include normal (SBP <120 mmHg and DBP <80 mmHg); elevated (SBP 120-129 mmHg and DBP <80 mmHg); hypertension stage 1 (SBP 130-139 mmHg or DBP 80-89 mmHg); hypertension stage 2 (SBP >140 mmHg or DBP >90 mmHg); hypertensive crisis (SBP >180 mmHg and/or DBP >120 mmHg).

These results demonstrates that dapagliflozin has the potential to reduce the risk of macrovascular complications such as Atherosclerotic Cardiovascular Disease (ASCVD) in prediabetes patients via its blood pressure lowering effects.

### Example 4: Dapagliflozin 5 mg Dose Rationale

A 5 mg dapagliflozin dose will be evaluated in the clinical study described in EXAMPLE 6. That dose was chosen, in part, based on the following rationale.

Pharmacokinetic/pharmacodynamic and maximum drug-induced effect models were previously developed to explore glucose-insulin dynamics in T2D patients participating in dapagliflozin Phase I-III clinical trials. Although the relationship is not linear, dapagliflozin treatment in T2D patients resulted in increased glucosuria, which correlated with decreases in HbA1c over time and it is expected that this correlation would extend through the HbA1c continuum for people with prediabetes.

To determine glucosuria in prediabetes patients, a modelling approach was applied. DAPA-HF and DAPA-CKD both included prediabetes populations that were randomized to dapagliflozin 10 mg. A modelling approach was used to predict the extent of 24-hour glucosuria in the prediabetes population in DAPA-HF and DAPA-CKD using 10 mg dapagliflozin, which in turn was used for estimating the efficacy/glycemic control of dapagliflozin 5 mg in prediabetes patients. The following approach was taken:

(1) Predict mean glucosuria in DAPA-CKD and DAPA-HF prediabetes populations taking dapagliflozin 10 mg using their HbA1c and eGFR values.

(2) Predict mean glucosuria in people with prediabetes taking dapagliflozin 5 mg at various eGFRs >45 ml/min/1.73 m².

(3) Compare glucosuria of DAPA-HF and DAPA-CKD pre-diabetics (10 mg dose) (from (1)) with that of pre-diabetics with an eGFR >45 ml/min/1.73 m² (5 mg dose) (from (2)).

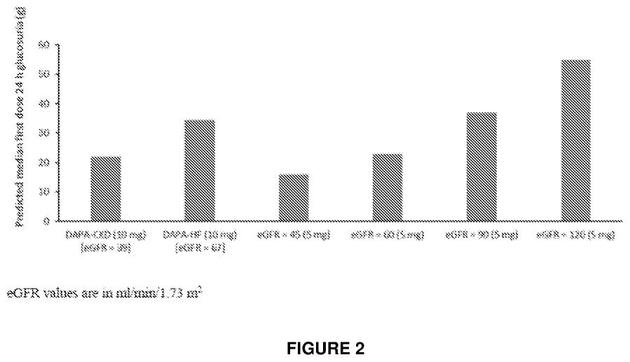

Notably, eGFR >45 ml/min/1.73 m² was selected as a criterion for glucosuria prediction in prediabetes patients because this is the current lower bound of eGFR for T2D in the dapagliflozin United States Prescribing Information (USPI). The results of the simulated glucosuria in patients with prediabetes in DAPA-CKD (10 mg), DAPA-HF (10 mg), and various eGFR values in people with prediabetes taking 5 mg dapagliflozin are illustrated in FIG. 2. Based on the glucosuria modelling, a once-daily 5 mg dapagliflozin dose in people with prediabetes with an eGFR of ≥60 ml/min/1.73 m² was predicted to provide a similar or somewhat better glucosuria and improved glycemia to that seen in the DAPA-CKD study (10 mg dapagliflozin once-daily).

### Example 5: Safety of Dapagliflozin 5 mg in Patients with Prediabetes

DAPA-HF and DAPA-CKD included patients without diabetes, including those who qualify as prediabetic. The results in this subgroup for Adverse Events (“AEs”) in any category were generally consistent with the overall safety results. Patients who did not have T2D at baseline generally reported fewer AEs than patients with T2D, which can be explained by the older age and generally greater morbidity in patients with T2D. Events of definite or probable diabetic ketoacidosis (“DKA”) and major hypoglycemic AEs were not observed in the non-diabetic and prediabetic patients.

All patients in DAPA-HF and DAPA-CKD were treated with 10 mg dapagliflozin or placebo once daily. The safety profile in the non-diabetic and prediabetic patients was consistent with the profile established in patients with T2D, with the exception that there were no events of DKA or hypoglycemia. The safety profile of dapagliflozin in diabetic and prediabetic patients is well-characterized based on the substantial safety database comprised of the pivotal diabetes trials, DECLARE, DAPA-HF, and DAPA-CKD results.

The results of the subgroup analyses by T2D status at baseline showed that the safety profile of dapagliflozin was similar to the overall results, regardless of T2D status at baseline. DKA and major hypoglycemic events were observed only in patients with T2D. Since DKA is a condition with severe insulin deficiency, events of DKA were not expected in the population without T2D (55% of the study population), including those with prediabetes, which was confirmed by the results. (See Umpierrez G, Korytkowski M. Diabetic emergencies-ketoacidosis, hyperglycaemic hyperosmolar state and hypoglycaemia. Nat Rev Endocrinol. 2016; 12 (4): 222-32.) The known adverse reactions are not dose-related, thus, it is expected that the safety profile of 5 mg dapagliflozin is consistent with that of 10 mg.

### Example 6: Overview of the Study Design

The study is designed to be an event-driven, randomized, double-blinded, placebo-controlled parallel-group, international multicenter study in participants with prediabetes to evaluate the effect of dapagliflozin 5 mg versus placebo given orally once daily to reduce the risk of developing T2D.

The target population includes male and female participants (≥45 years) with prediabetes at risk of developing T2D, and without HF or CKD. Approximately 4800 participants will be randomized to study treatment. Randomization may be capped on proportion of prediabetes participants in the 5.7%≤HbA1c <6.0% range. Laboratory parameters (e.g., HbA1c, creatinine/eGFR, UACR), vital signs, and AEs will be collected at randomization and then every three months through to study closeout. Safety and tolerability will be evaluated in terms of Serious Adverse Events (SAEs) and Adverse Events (AEs) leading to discontinuation of study drug.

The primary efficacy endpoint will be time from randomization to the new onset of T2D based on two HbA1c values above the cut-off level for diabetes (HbA1c≥6.5%). Participants with HbA1c≥6.5% will be required to provide a follow-up blood sample and a second elevated HbA1c will confirm the diagnosis of T2D. A diagnosis of T2D can also be based on patients achieving the diagnosis and/or treatment with oral diabetic medicines outside the study. Participants who develop T2D during the treatment period will be eligible for rescue therapy according to the local standard of care (excluding SGLT2 inhibitors). Participants who receive rescue therapy will remain in the study.

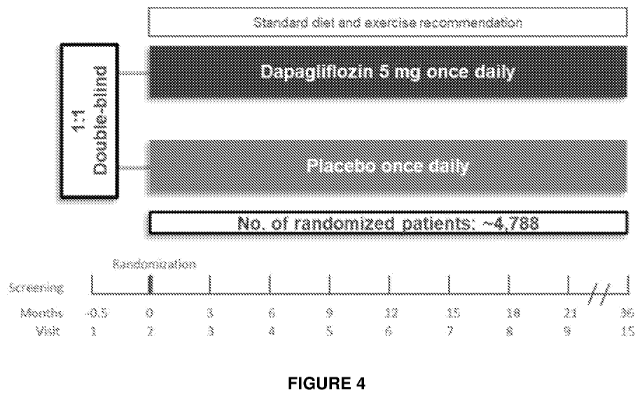

One of the secondary efficacy endpoints is the rate of change in eGFR over time from 3 months after randomization until the end of the study (i.e., eGFR chronic slope). Participants without already known kidney disease will be enrolled. The Sponsor, Investigator, and participants will be blinded to eGFR and UACR and the Investigator will assume the necessary standard of care for the participant where needed. The study design is further illustrated in FIG. 4 and Table 3.

TABLE 3
Study Assessment

| | Activity | | | | | | | | |
|---|---|---|---|---|---|---|---|---|---|
| | Enrolment | Randomization | Site visits | | | | | | |
| | Visit number | | | | | | | | |
| | | 2 | 3 | 4 | 5 | 6 | 7, 8 etc | | |
| | | Day | | | | | | | |
| | | | | | | | Every | | |
| | | | 90 | 180 | 270 | 360 | 3^{rd} | | |
| | 1 | 0 | (±7) | (±7) | (±7) | (±7) | month | PTDV^{a} | SCV^{b} |
| Signed Informed | X | X | | | | | | | |
| Consent Form | | | | | | | | | |
| Demography | X | | | | | | | | |
| Medical history | X | | | | | | | | |
| Inclusion/exclusion | X | X | | | | | | | |
| criteria | | | | | | | | | |
| General Physical | X | | | | | | | | |
| examination | | | | | | | | | |
| Height | X | | | | | | | | |
| Vital signs (BP, | X | X | X | X | X | X | X | X | X |
| pulse and body | | | | | | | | | |
| weight) | | | | | | | | | |
| Concomitant | X | X | X | X | X | X | X | X | X |
| medication | | | | | | | | | |
| Pregnancy | X | | | | | | | | |
| testing^{c} | | | | | | | | | |
| HbA1c^{d} | X | X | X | X | X | X | X | X | X |
| Creatinine/eGFR^{d} | | X | X | X | X | X | X | X | X |
| UACR^{d} | | X | X | X | X | X | X | X | X |
| AE collection^{e} | | X | X | X | X | X | X | X | X |
| Sample for | | X | X | X | | X | | | |
| biomarker | | | | | | | | | |
| research, if | | | | | | | | | |
| applicable^{f} | | | | | | | | | |

^{a}Patients who prematurely and permanently discontinue treatment with study medication should return for a Premature Treatment Discontinuation Visit (PTDV). Patients who discontinue treatment prematurely still need to attend a Study Closure Visit
^{b}The Study Closure Visit (SCV)
^{c}Only applicable for women of childbearing potential (i.e., those who are not chemically or surgically sterilised or post-menopause)
^{d}HbA1c, creatinine/eGFR, and UACR will be assessed at a central lab. Creatinine/eGFR and UACR lab results will be kept blinded to patients, investigators and sponsor during study.
^{e}Serious AE and AEs leading to discontinuation of study drug
^{f}Serum, plasma and urine samples for future biomarker research will be collected if applicable and will be analysed in line with specifications in the clinical study protocol and informed consent form at the discretion of the sponsor. The Biomarker sampling is optional and subject to separate approval/consent by the patient at Visit 1.

### Study Population

This study subjects will include overweight male and female adults with prediabetes and at least one additional established risk factor for T2D. Key inclusion and exclusion criteria are presented in Table 4.

TABLE 4
Inclusion and Exclusion Criteria

Inclusion Criteria
1 Male or female, aged ≥45 years at the time of consent
2 BMI ≥25 kg/m2
5.7% ≤ HbA1c < 6.5% confirmed by two measurements
from two separate samples at the screening visit)
4 At least one of the following risk factors:
BMI ≥30 kg/m2
First degree relative with T2D
Medical history of hypertension
Medical history of dyslipidaemia
Prior gestational diabetes
Polysystic ovary syndrome
5 Prior unsuccessful attempt with diet and exercise
Exclusion Criteria
6 Participants with known T2D
Participants with known Type 1 diabetes mellitus,
latent autoimmune diabetes in adult, maturity onset
diabetes in young
8 Participants with known CKD
9 Participants with known HF
For women only-currently pregnant confirmed with
positive pregnancy test or breastfeeding, or are
planning a pregnancy
Receiving therapy with an SGLT2 inhibitor and any
other glucose lowering medicines within 12 weeks
prior to enrolment or previous intolerance of an
SGLT2 inhibitor

### Objectives and Endpoints

Table 5 outlines objectives and endpoints of the study.

TABLE 5
Objectives and Endpoints

| | |
|---|---|
| Objective | Endpoint |

Primary objective

| | |
|---|---|
| To determine whether dapagliflozin 5 mg is | Time to new onset of T2D |
| superior to placebo in reducing the risk of | (HbA1c ≥ 6.5) |
| developing T2D | |

Secondary objectives

| | |
|---|---|
| To compare the effect of treatment of | Rate of change in eGFR |
| dapagliflozin versus placebo on eGFR | over time (ie eGFR chronic |
| chronic slope | slope) from 3 months after |
| | randomization until end of |
| | study |
| To compare the effect of treatment of | Change in blood pressure |
| dapagliflozin versus placebo on blood | from baseline to Week 24 |
| pressure | |
| To compare the effect of treatment of | Change in body weight |
| dapagliflozin versus placebo on body | from baseline to Week 24 |
| weight | |

Safety objective

| | |
|---|---|
| To assess the safety and tolerability | Safety and tolerability will |
| of dapagliflozin as compared to placebo | be evaluated in terms of |
| in participants with prediabetes. | SAEs and AEs leading to |
| | discontinuation of study drug. |

### Primary Objective

The primary objective of the study is to determine the superiority of dapagliflozin versus placebo in reducing the risk of T2D. Assuming a true hazard ratio of 0.75 between dapagliflozin and placebo, using a one-sided alpha of 2.5%, 508 primary endpoint events will provide a statistical power of 90% for the test of the primary endpoint. This is based on an overall 1:1 allocation between dapagliflozin and placebo. With an annual event rate of 6.0% in the placebo treatment group, 4788 participants (i.e., 2394 per arm) are estimated to provide the required number of primary endpoint events, based on an anticipated recruitment period of 1.5 years and a maximum follow-up period of approximately 3 years with an overall dropout of 10%.

### Secondary Objectives

The secondary objective of eGFR chronic slope is to compare the effect of treatment of dapagliflozin versus placebo on eGFR slope from 3 months to end of study to assess microvascular benefit. The 4788 participants (2394 per arm) planned for the study will provide >80% power to detect a 0.5 ml/min/1.73 m² difference in eGFR slope between dapagliflozin and placebo at a significance level of 0.025 (one-sided) when assuming a common standard deviation of 6 ml/min/1.73 m². The assumed common standard deviation of 6 ml/min/1.73 m² is based on the estimates from the DECLARE eGFR slope analyses in the subgroup close to the planned prediabetes population (T2D <5 years or HbA1c <7% and other factors). The group difference of 0.5 ml/min/1.73 m² in eGFR slope would be considered to be clinically meaningful in early stages of CKD and the expected eGFR decline in the prediabetes stage is at that of early CKD. (Levey et al., Change in Albuminuria and GFR as End Points for Clinical Trials in Early Stages of CKD. Am J Kidney Dis. 2020; 75 (1): 84-104.)

The secondary efficacy endpoint analysis on eGFR chronic slope will compare dapagliflozin 5 mg with placebo based on the difference between the treatment groups in eGFR chronic slopes using a mixed-effects model in Full Analysis Set (FAS). The model will include fixed effects for treatment group, baseline HbA1c range stratification status, baseline eGFR, time, and time-by-treatment interaction. Random effects will include the intercept and the slope (3 months to end of study). End of study is the date the pre-defined target number of events for the primary endpoint occurs.

The other secondary objectives compare the effect of treatment of dapagliflozin versus placebo on blood pressure and body weight as ways of assessing macrovascular benefit. The sample size will also provide adequate power to assess the effect of treatment of dapagliflozin versus placebo on the secondary efficacy endpoints of blood pressure and body weight.

### Example 7: Development of the Web App Program

A combination product with a medical device in the form of a Web App will be developed. Under this program, consumer access will be limited to online purchase after the consumer has qualified (and requalified on a subsequent purchase) via the Web App by answering predetermined questions corresponding to the elements in the Drug Facts Label (DFL). Using the Web App, coupled with online access that requires an assessment of a consumer's self-selection, is required prior to each purchase.

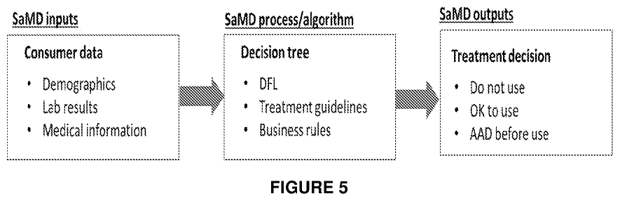

The Web App features a technology-assisted self-selection (TASS) tool, which has software-as-a-medical-device (SaMD) functions. Specifically, the Web App is considered SaMD because of the diagnosis and treatment decision provided by the TASS. The TASS provides a determination as to whether or not the product is right for the consumer. FIG. 5 outlines the SaMD inputs, process, and outputs that lead to 1 of 3 treatment decisions (i.e., Do Not Use, OK to Use, or Ask A Doctor Before Use).

The technology-assisted self-selection (TASS) acts as a digitized, interactive version of the DFL, allowing consumers to answer a dynamic and personalized set of health-related questions, in lieu of browsing/searching a DFL for information pertinent to them. This may ensure that only the consumers who will benefit from the medication receive it. If the consumer qualifies based on use of the Web App, he or she would purchase the product online from the website. The product would only be available to consumers who had an “OK to use” and decided to purchase via the website. The product would then be shipped to consumers with a valid US address. Before repurchasing the product, a consumer would have to answer some additional questions on any changes to their medical status before they would be requalified for purchase.

1. A method of reducing the risk of developing Type 2 diabetes in a patient in need thereof, the method comprising administering to the patient, an effective amount of a sodium-glucose co-transporter 2 (SGLT2) inhibitor, wherein the patient has an HbA1c between 5.7% to 6.4% and/or a fasting glucose between 100 to 125 mg/dl.

2. A method of treating prediabetes in a patient in need thereof, the method comprising administering to the patient, an effective amount of a SGLT2 inhibitor, wherein the patient has an HbA1c between 5.7% to 6.4% and/or a fasting glucose between 100 to 125 mg/dl.

3. The method according to any one of claims 1 to 2, wherein the patient does not have Type 1 diabetes (T1D) or Type 2 diabetes (T2D).

4. The method according to any one of claims 1 to 3, wherein the patient was not previously administered a prescription medicine for diabetes.

5. The method according to any one of claims 1 to 4, wherein the patient does not have chronic kidney disease (CKD) and/or heart failure (HF).

6. The method according to any one of claims 1 to 5, wherein the SGLT2 inhibitor is chosen from dapagliflozin, canagliflozin, empagliflozin, sotagliflozin, ipragliflozin, ertugliflozin, tofogliflozin, and luseogliflozin, or a pharmaceutically acceptable salt, solvate, mixed solvate, complex, or prodrug of any of the foregoing.

7. The method according to any one of claims 1 to 6, wherein the SGLT2 inhibitor is dapagliflozin.

8. The method according to claim 7, wherein dapagliflozin has the structure

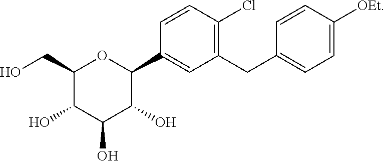 [SMILES: CCOc1ccc(Cc2cc([C@@H]3O[C@H](CO)[C@@H](O)[C@H](O)[C@H]3O)ccc2Cl)cc1]

9. The method according to any one of claims 7 to 8, wherein the dapagliflozin is in the form of a pharmaceutically acceptable solvate, mixed solvate, or complex.

10. The method according to any one of claims 7 to 9, wherein the dapagliflozin is in the form of a non-crystalline solid.

11. The method according to any one of claims 7 to 10, wherein the dapagliflozin is in the form of a crystalline solid.

12. The method according to any one of claims 7 to 11, wherein the dapagliflozin is in the form of a (S)-propylene glycol ((S)-PG) solvate.

13. The method according to claim 12, wherein the(S)-propylene glycol ((S)-PG) solvate has the structure

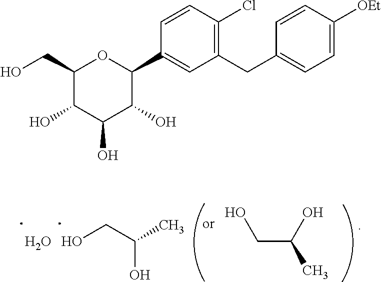

14. The method according to any one of claims 7 to 13, wherein the dapagliflozin is administered orally.

15. The method according to claim 14, wherein the dapagliflozin is in the form of a tablet.

16. The method according to any one of claims 7 to 15, wherein the dapagliflozin is administered in a dosage of 2.5 mg/day, 5 mg/day, or 10 mg/day.

17. The method according to claim 16, wherein the dapagliflozin is administered in a dosage of 2.5 mg/day.

18. The method according to claim 16, wherein the dapagliflozin is administered in a dosage of 5 mg/day.

19. The method according to any one of claims 7 to 18, wherein the dapagliflozin is administered once daily.

20. The method according to any one of claims 7 to 19, wherein the dapagliflozin is administered in combination with at least one other therapeutic agent.

21. The method according to claim 20, wherein the at least one other therapeutic agent is chosen from an antidiabetic agent, anti-obesity agent, anti-hyperlipidemic agent, anti-atherosclerotic agent, anti-hypertensive agent, anti-platelet agent, antithrombotic agent, mineralocorticoid antagonist, diuretic, and anticoagulant agent.

22. The method according to claim 20, wherein the at least one other therapeutic agent is an angiotensin-converting enzyme inhibitor (ACE inhibitor).

23. The method according to claim 22, wherein the ACE inhibitor is chosen from captopril, enalapril, and lisinopril.

24. The method according to claim 20, wherein the at least one other therapeutic agent is an angiotensin receptor blocker (ARB).

25. The method according to claim 24, wherein the ARB is chosen from valsartan, losartan, and irbesartan.

26. The method according to any one of claims 20 to 25, wherein the at least one other therapeutic agent is administered before, after, or concurrently with dapagliflozin.

27. The method according to any one of claims 1 to 26, wherein prior to the administration, the patient had an eGFR of ≥39 and ≤67 mL/min/1.73 m².

28. The method according to any one of claims 1 to 27, wherein prior to the administration, the patient had an eGFR of ≥60 mL/min/1.73 m².

29. The method according to any one of claims 1 to 28, wherein the method results in a relative risk reduction of 25% or more for developing T2D.

30. The method according to claim 29, wherein the method results in a relative risk reduction of 30% for developing T2D.

31. The method according to any one of claims 1 to 30, wherein the method results in an absolute risk reduction of 3% or more for developing T2D during a period of 1.75 years.

32. The method according to any one of claims 29 to 31, wherein the method results in a hazard ratio from 0.65 to 0.8.

33. The method according to claim 32, wherein the hazard ratio is 0.75, 0.72 or 0.69.

34. The method according to any one of claims 1 to 33, wherein the patient satisfies at least one of the following conditions:
(l) the patient has a BMI of ≥30 kg/m²;
(m) the patient has a first degree relative with T2D;
(n) the patient has a medical history of hypertension;
(o) the patient has a medical history of dyslipidemia;
(p) the patient has prior gestational diabetes; and/or
(q) the patient has polycystic ovary syndrome.

35. The method according to any one of claims 1 to 34, wherein the patient is ≥45 years old and has a body mass index of ≥30 kg/m² for non-Asians or >27 kg/m² for Asians.

36. The method according to any one of claims 1 to 35, wherein the patient satisfies one or more of the following conditions:
(f) the patient does not have a fasting plasma glucose of ≥7 mmol/L;
(g) the patient does not have T2D;
(h) the patient does not have HF;
(i) the patient does not have CKD stage 3 to 5; and/or
(j) the patient does not have severe hepatic impairment of Child-Pugh class 3.

37. The method according to any one of claims 1 to 36, wherein prior to the administration, the patient accessed a webpage and provided answers to predetermined questions; and the patient was determined to be qualified to purchase the SGLT2 inhibitor based on the provided answers.

38. The method according to any one of claims 1 to 37, wherein the administration does not require a medical prescription.

39. The method according to any one of claims 1 to 38, wherein the administration reduces the risk of developing microvascular and/or macrovascular complications.

40. The method according to claim 39, wherein the administration results in a relative risk reduction of 28% for developing microvascular complications.

41. The method according to claims 1 to 40, wherein the administration reduces blood pressure.

42. The method according to claims 1 to 41, wherein the administration reduces body weight.
